(19) United States
(12) Patent Application Publication
(10) Pub. No.: US 2007/0199159 A1
(43) Pub. Date: Aug. 30, 2007

(54) Dye-containing pellets and their use

(76) Inventors: Jurgen Schmenger, Weiterstadt (DE); Petra Braun, Munster (DE)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/705,626

(22) Filed: Feb. 12, 2007

(30) Foreign Application Priority Data
Feb. 13, 2006 (EP) 06002861.0

(51) Int. Cl. A61K 8/00

(52) U.S. Cl. 8/405

(57) ABSTRACT

The present patent application relates to dye-containing pellets obtained by (a) homogeneous mixing of a starting material containing at least one natural and/or synthetic dye with a suitable carrier material and then coating with a suitable encapsulation material or (b) coating a suitable carrier material with a mixture of at least one natural and/or synthetic dye and at least one suitable encapsulation material, to the use of said pellets for preparing colorants for keratin fibers and to the colorants based on said pellets. Furthermore the present patent application relates to the use of these pellets for increasing the shine of the keratin fibers, reducing the scalp coloring of hair dyes, improving the protection for the cortex, increasing the colorant efficiency and reducing the load of colorants, and improving the wash resistance.

### FIELD OF THE INVENTION

The present invention relates to dye-containing pellets obtained by a special method of preparation, as well as the use of said pellets for coloring keratin fibers.

### BACKGROUND OF THE INVENTION

Direct dyes, nitro dyes and pigment dyes or oxidation dyes in the form of colorless developer/coupler precursors are the dyes usually employed for the coloring of keratin fibers.

Dyes are sold in conventional application forms. These application forms vary from liquid to creamy to waxy products. Aerosols, for example, foam hair dyes, are also in use. Powdered dyes that must be mixed with an aqueous medium before use are also on the market.

The aforesaid agents, however, are not satisfactory in every respect. For example, in the case of oil-treated, dust-free powders, the oils used have an adverse effect on product performance, whereas when reactive dyes and raw materials are used as well as in the case of powders and liquid systems, problems arise in terms of storage stability.

### SUMMARY OF THE INVENTION

By means of a suitable method that is carried out with the aid of suitable carrier and encapsulation (coating) materials, a dye-containing pellet for coloring keratin fibers which does not have the aforedescribed drawbacks and provides better dosability and multicolor effects.

### DETAILED DESCRIPTION OF THE INVENTION

The present patent application relates to a dye-containing pellet which is obtained by (a) homogeneous mixing of a starting material containing at least one natural and/or synthetic dye with a suitable carrier material and then coating with a suitable encapsulation material or (b) coating a suitable carrier material with a mixture of at least one natural and/or synthetic dye and at least one suitable encapsulation material.

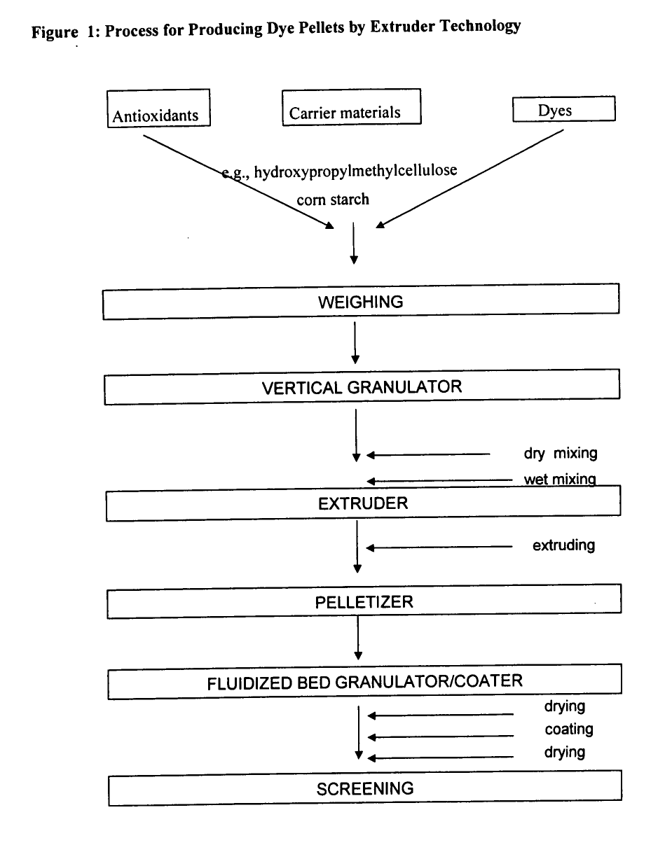

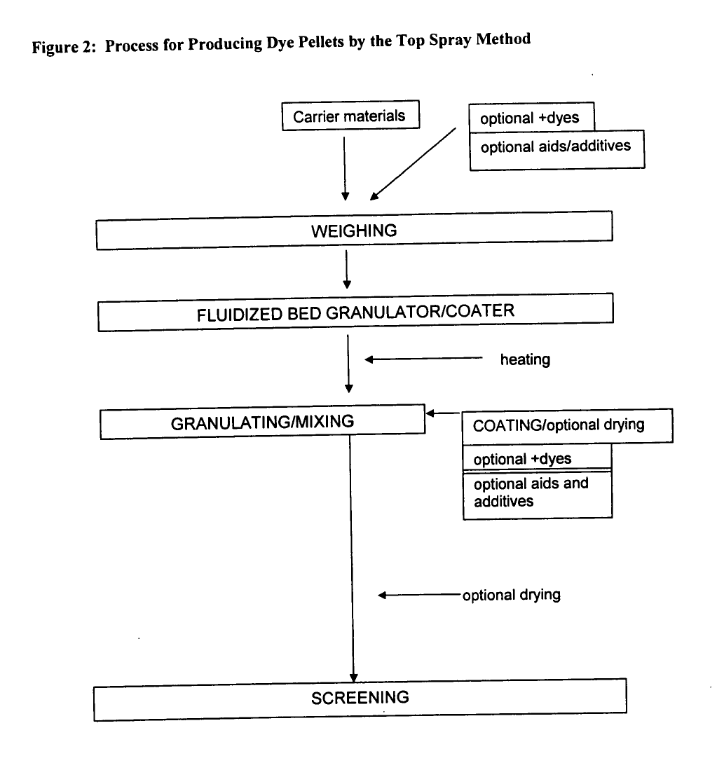

The dye-containing pellets of the invention can be prepared by the process based on extruder technology and schematically shown in FIG. 1 [pellets as under (a)] or by the top spray process [pellets as under (a) or (b)] schematically presented in FIG. 2.

1. Preparation Based on Extruder Technology

A base composition is prepared in a vertical granulator (rotor rotational speed=50 rpm to 200 rpm and preferably 150 rpm; chopper rotational speed=750 rpm to 1250 rpm and preferably 1000 rpm) at room temperature (15° C. to 35° C.) by dry mixing and then wet mixing the dye composition with a carrier material and, optionally, antioxidants and other auxiliary agents. This base composition is then extruded from an extruder (rotational speed=15 rpm to 50 rpm and preferably 25 rpm to 30 rpm; screen mesh size=0.01 mm to 5 mm, preferably 0.1 mm to 3 mm and particularly 0.6 mm to 1 mm). The resulting granulate is rounded in a pelletizer (rotational speed=400 rpm to 800 rpm and preferably 500 rpm to 600 rpm). The granulate is then dried at a product temperature of 20° C. to 60° C. (preferably 30° C. to 55° C.) (incoming air temperature preferably 70° C. to 80° C.) and then (optionally after previous warming to 40° C. to 50° C.) coated by the fluidized bed process (spraying rate preferably 5 g/min. to 20 g/min.; spraying air pressure preferably 1.5 bar to 2.5 bar), the quantity of encapsulation material (based on the quantity of granulate to be coated) amounting to 0.5 wt. % to 50 wt. %, preferably 1 wt. % to 20 wt. % and particularly 2 wt. % to 15 wt. %. The product is finally dried (maximum product temperature 51° C.).

2. Preparation by the Top Spray Process

(a) By this process, the dye composition is mixed with the carrier materials and optionally with antioxidants and other auxiliary agents in a fluidized bed granulator/coater (rotor rotational speed=50 rpm to 200 rpm and preferably 150 rpm); chopper rotational speed=750 rpm to 1250 rpm and preferably 1000 rpm) at room temperature (15° C. to 35° C.). The base composition thus obtained is then heated (maximum product temperature 34° C.), then granulated and finally coated (spraying rate preferably 6 g/min. to 20 g/min.; spraying air pressure preferably 0.25 bar to 0.75 bar), the quantity of encapsulation material used (based on the quantity of the granulate to be coated) amounting to 0.5 wt. % to 50 wt. %, preferably 1 wt. % to 20 wt. % and particularly 2 wt. % to 10 wt. %. If necessary, the product is then dried (maximum product temperature 60° C.).

(b) The carrier materials and optionally the antioxidants and other auxiliary agents are mixed with one another in a fluidized bed granulator/coater (rotor rotational speed=50 rpm to 200 rpm and preferably 150 rpm; chopper rotational speed=750 rpm to 1250 rpm and preferably 1000 rpm) at room temperature (15° C. to 35 ° C.). The base composition thus obtained is then heated (maximum product temperature 34° C.), then granulated and finally coated with a solution/dispersion of the dyes in a suitable encapsulation material (spraying rate preferably 6 g/min. to 20 g/min.; spraying air pressure preferably 0.25 bar to 0.75 bar), the quantity of encapsulation material used (based on the quantity of granulate to be coated) amounting to 0.5 wt. % to 50 wt. %, preferably 1 wt. % to 20 wt. % and particularly 2 wt. % to 10 wt. %. If necessary, the product is then dried (maximum product temperature 57° C.).

Suitable carrier materials for the dye-containing pellets are powdered, microcrystalline substances which place the dye in a physical state that allows the process for the coating of the pellets with suitable encapsulation materials to be carried out. Suitable carrier materials are, in particular, polyvinylpyrrolidone, dextrose, oligosaccharides, microcrystalline cellulose derivatives, for example, hydroxypropylmethylcellulose, methylcellulose, hydroxyethylcellulose, hydroxypropyl-cellulose, nonoxynol-hydroxyethylcellulose and cetylhydroxyethylcellulose, or physically or che-mically modified starches or starch derivatives, for example, starch esters (for example, acetylated starches), starch ethers (for example, hydroxyalkylated starches), dialdehyde starches, dicar-boxylstarches, distarch phosphates, hydroxyalkylstarch phosphates or hydroxyalkyl starches, wherein the alkyl groups preferably contain from 1 carbon atom to 4 carbon atoms and more preferably 2 carbon atoms to 3 carbon atoms. Also suitable are crosslinked starch ethers, for example, those bearing the INCI designations dimethylimidazolidone rice or corn starch, or hydrophobically modified starches (for example those bearing the INCI designation aluminum starch octenesuccinates). The starch can be modified thermally, hydrolytically or enzymatically, the starting starch possibly being obtained from known sources, for example, corn, potatoes, sweet potatoes, peas, bananas, oats, wheat, barley, rice, sago, tapioca, taproot, amaranth, canna, sorghum etc. Particularly preferred starch derivatives are the nonionic starch derivatives, particularly the nonionic starch derivatives modified with an alkylene oxide such as ethylene oxide, propylene oxide or butylene oxide, or with acetic anhydride or butyl ketene dimer, and particularly with propylene oxide. Other suitable carrier materials are synthetic calcium silicate, diatomite, silicone dioxide or other free-flowing, non-caking powders.

Suitable encapsulation materials for the pellets of the invention are water-soluble or water-dispersible, film-forming substances which when spray-dried from solutions or dispersions are capable of depositing uniform films so that one can speak about encasing (coating). Suitable encapsulation materials are cellulose derivatives (for example, methylcelluloses), polyethylene dispersions, polyacrylic acids, polyvinyl alcohols, polycarbonates, polyvinylpyrrolidone, polyesters and polyamides or natural film-formers, for example, chitosan, shellac, oligosaccharides or Chinese balsam resin (colophony).

Suitable oxidation dye precursors are, for example, the following developers, couplers and self-coupling substances.

(i) Developers: 1,4-diaminobenzene (p-phenylenediamine), 1,4-diamino-2-methylbenzene (p-toluylenediamine), 1,4-diamino-2,6-dimethylbenzene, 1,4-diamino-3,5-di-ethylbenzene, 1,4-diamino-2,5-dimethylbenzene, 1,4-diamino-2,3-dimethylbenzene, 2-chloro-1,4-diaminobenzene, 1,4-diamino-2-(thiophen-2-yl)benzene, 1,4-diamino-2-(thiophen-3-yl)benzene, 1,4-diamino-2-(pyridin-3-yl)benzene, 2,5-diaminobiphenyl, 1,4-diamino-2-methoxymethylbenzene, 1,4-diamino-2-aminomethylbenzene, 1,4-diamino-2-hydroxymethylbenzene, 1,4-diamino-2-(2-hydroxyethoxy)benzene, 2-[2-(acetylamino)ethoxy]-1,4-diaminobenzene, 4-phenylaminoaniline, 4-dimethylaminoaniline, 4-diethylaminoaniline, 4-dipropylaminoaniline, 4-[ethyl-(2-hydroxyethyl)amino]aniline, 4-[di(2-hydroxyethyl)amino]aniline, 4-[di(2-hydroxyethyl)amino]-2-methylaniline, 4-[(2-methoxyethyl)amino]aniline, 4-[(3-hydroxypropyl)amino]aniline, 4-[(2,3-dihydroxypropyl)amino]-aniline, 1,4-diamino-2-(2-hydroxyethyl)benzene, 1,4-di-amino-2-(1-methylethyl)benzene, 1,3-bis[(4-aminophenyl)-(2-hydroxyethyl)amino]-2-propanol, 1,4-bis[(4-aminophenyl)amino]butane, 1,8-bis(2,5-di-aminophenoxy)-3,6-dioxaoctane, 4-aminophenol, 4-amino-3-methylphenol, 4-amino-3-(hydroxymethyl)phenol, 4-amino-3-fluorophenol, 4-methylaminophenol, 4-amino-2-(aminomethyl)phenol, 4-amino-2-(hydroxymethyl)phenol, 4-amino-2-fluorophenol, 4-amino-2-[(2-hydroxyethyl)amino]methylphenol, 4-amino-2-methylphenol, 4-amino-2-(methoxymethyl)phenol, 4-amino-2-(2-hydroxyethyl)phenol, 5-aminosalicylic acid, 2,5-diaminopyridine, 2,4,5,6-tetra-aminopyrimidine, 2,5,6-triamino-4-(1H)-pyrimidone, 4,5-diamino-1-(2-hydroxyethyl)-1H-pyrazole, 4,5-diamino-1-(1-methylethyl)-1H-pyrazole, 4,5-diamino-1-[(4-methylphenyl)methyl]-1H-pyrazole, 1-[(4-chlorophenyl)methyl]-4,5-diamino-1H-pyrazole, 4,5-diamino-1-methyl-1H-pyrazole, 2-ami-nophenol, 2-amino-6-methyl-phenol and 2-amino-5-methylphenol, or in mixtures thereof.

(ii) Couplers: N-(3-dimethylaminophenyl)urea, 2,6-diaminopyridine, 2-amino-4-[(2-hydroxyethyl)amino]anisole, 2,4-diamino-1-fluoro-5-methylbenzene, 2,4-diamino-1-methoxy-5-methylbenzene, 2,4-diamino-1-ethoxy-5-methylbenzene, 2,4-diamino-1-(2-hydroxyethoxy)-5-methylbenzene, 2,4-di[(2-hydroxyethyl)amino]-1,5-dimethoxybenzene, 2,3-diamino-6-methoxypyridine, 3-amino-6-methoxy-2-(methylamino)pyridine, 2,6-diamino-3,5-dimethoxypyridine, 3,5-diamino-2,6-dimethoxypyridine, 1,3-diaminobenzene, 2,4-diamino-1-(2-hydroxyethoxy)benzene, 1,3-diamino-4-(2,3-dihydroxypropoxy)benzene, 2,4-diamino-1,5-di(2-hydroxyethoxy)benzene, 1-(2-aminoethoxy)-2,4-diaminobenzene, 2-amino-1-(2-hydroxyethoxy)-4-methylaminobenzene, 2,4-diaminophenoxyacetic acid, 3-[di(2-hy-droxyethyl)amino]aniline, 4-amino-2-di[(2-hydroxyethyl)amino]-1-ethoxybenzene, 5-methyl-2-(1-methylethyl)phenol, 3-[(2-hydroxyethyl)amino]aniline, 3-[(2-aminoethyl)amino]aniline, 1,3-di(2,4-diaminophenoxy)propane, di(2,4-diaminophenoxy)methane, 1,3-diamino-2,4-dimethoxybenzene, 2,6-bis(2-hydroxyethyl)aminotoluene, 4-hydroxyindole, 3-dimethylaminophenol, 3-diethylaminophenol, 5-amino-2-methylphenol, 5-amino-4-fluoro-2-methylphenol, 5-amino-4-methoxy-2-methylphenol, 5-amino-4-ethoxy-2-methylphenol, 3-amino-2,4-dichlorophenol, 5-amino-2,4-dichlorophenol, 3-amino-2-methylphenol, 3-amino-2-chloro-6-methylphenol, 3-aminophenol, 2-[(3-hydroxyphenyl)amino]acetamide, 5-[(2-hydroxyethyl)amino]4-methoxy-2-methylphenol, 5-[(2-hydroxyethyl)amino]-2-methylphenol, 3-[(2-hydroxyethyl)amino]-phenol, 3-[(2-methoxyethyl)amino]phenol, 5-amino-2-ethylphenol, 5-amino-2-methoxyphenol, 2-(4-amino-2-hydroxyphenoxy)ethanol, 5-[(3-hydroxypropyl)amino]-2-methylphenol, 3-[(2,3-dihydroxypropyl)amino]-2-methylphenol, 3-[(2-hydroxyethyl)amino]-2-methylphenol, 2-amino-3-hydro-xypyridine, 5-amino-4-chloro-2-methylphenol, 1-naphthol, 2-methyl-1-naphthol, 1,5-dihydroxy-naphthalene, 1,7-dihydroxynaphthalene, 2,3-dihydroxynaphthalene, 2,7-dihydroxynaphthalene, 2-methyl-1-naphthol acetate, 1,3-dihydroxybenzene, 1-chloro-2,4-dihydroxybenzene, 2-chloro-1,3-dihydroxybenzene, 1,2-dichloro-3,5-dihydroxy-4-methylbenzene, 1,5-dichloro-2,4-dihydroxybenzene, 1,3-dihydroxy-2-methylbenzene, 3,4-methylene-dioxyphenol, 3,4-methylenedioxyaniline, 5-[(2-hydroxyethyl)amino]-1,3-benzodioxole, 6-bromo-1-hydroxy-3,4-methylenedioxybenzene, 3,4-diaminobenzoic acid, 3,4-dihydro-6-hydroxy-1,4(2H)-benzoxazine, 6-amino-3,4-dihydrol,4(2H)-benzoxazine, 3-methyl-1-phenyl-5-pyrazolone, 5,6-dihydroxyindole, 5,6-dihydroxyindoline, 5-hydroxyindole, 6-hydroxyindole, 7-hydroxyindole and 2,3-indolinedione, or in mixtures thereof.

(iii) Self-coupling compounds: 2-amino-5-methylphenol, 2-amino-6-methylphenol, 2-amino-5-ethoxyphenol or 2-propylamino-5-aminopyridine.

Among the aforesaid oxidation dye precursors, the following compounds, alone or in combination with one another, are particularly preferred: 2,5-diaminotoluene, 2,4-diaminophenoxyethanol, resorcinol, 2-methylresorcinol, m-aminophenol, 4-amino-m-cresol, 4-amino-2-hydroxytoluene, 6-amino-m-cresol, 2-amino-4-hydroxyethylaminoanisol, 1-naphthol, hydroxyethyl-3,4-methylene-dioxyaniline, 2,5-diaminophenylethanol, N,N-bis(2-hydroxyethyl)-p-phenylenediamine, phenyl-methylpyrazolone, 1-hydroxyethyl-4,5-diaminopyrazole and 2-amino-6-chloro-4-nitrophenol or the salts thereof.

The total quantity of oxidation precursors present in the pellets of the invention amounts to 0.1 wt. % to 70 wt. % and particularly 0.5 wt. % to 50 wt. %.

To achieve certain color shades, it is also possible to use common natural and/or synthetic direct dyes, for example, vegetable dyes such as henna or indigo, triphenylmethane dyes, aromatic nitro dyes, azo dyes, quinone dyes, cationic dyes, or anionic dyes.

It is also possible for the pellets of the invention to contain exclusively direct dyes, namely without added oxidation precursors.

Suitable synthetic dyes are, for example: hydroxyethyl-2-nitro-p-toluidine, 2-hydroxyethylpicramic acid, 4-nitrophenylaminourea, tri(4-amino-3-methylphenyl)carbenium chloride (Basic Violet 2), 1,4-diamino-9,10-anthracenedione (Disperse Violet 1), 1,4-bis[(2-hydroxyethyl)amino]-2-nitro-benzene, 1-(2-hydroxyethyl)amino-2-nitro-4-[di(2-hydroxyethyl)amino]benzene (HC Blue No. 2), 1-amino-3-methyl-4-[(2-hydroxyethyl)amino]-6-nitrobenzene (HC Violet No. 1), 4-[ethyl-(2-hydro-xyethyl)amino]-1-[(2-hydroxyethyl)amino]-2-nitrobenzene hydrochloride (HC Blue No. 12), 4-[di(2-hydroxyethyl)amino]-1-[(2-methoxyethyl)amino]-2-nitrobenzene (HC Blue No. 11), 1-[(2,3-dihy-droxypropyl)amino]4-[methyl-(2-hydroxyethyl)amino]-2-nitrobenzene (HC Blue No. 10), 1-[(2,3-dihydroxypropyl)amino]4-[ethyl-(2-hydroxyethyl)amino]-2-nitrobenzene hydrochloride (HC Blue No. 9), 1-(3-hydroxypropylamino)-4-[di(2-hydroxyethyl)amino]-2-nitrobenzene (HC Violet No. 2), 1-methylamino-4-[methyl-(2,3-dihydroxypropyl)amino]-2-nitrobenzene (HC Blue No. 6), 2-[(4-ami-no-2-nitrophenyl)amino]-5-dimethylaminobenzoic acid (HC Blue No. 13), 1-amino-4-[(2-hydroxy-ethyl)amino]-2-nitrobenzene (HC Red No. 7), 2-amino-4,6-dinitrophenol, 4-amino-2-nitro-diphenylamine (HC Red No. 1), 1-amino4-[di(2-hydroxyethyl)amino]-2-nitrobenzene hydrochloride (HC Red No. 13), 1-amino-5-chloro-4-[(2-hydroxyethyl)amino]-2-nitrobenzene, 4-amino-1-[(2-hydroxyethyl)amino]-2-nitrobenzene (HC Red. No. 3), 4-amino-3-nitrophenol, 4-[(2-hydroxyethyl)amino]-3-nitrophenol, 1-[(2-aminoethyl)amino]4-(2-hydroxyethoxy)-2-nitrobenzene (HC Orange No. 2), 4-(2,3-dihydroxypropoxy)-1-[(2-hydroxyethyl)amino]-2-nitrobenzene (HC Orange No. 3), 1-amino-5-chloro4-[(2,3-dihydroxypropyl)amino]-2-nitrobenzene (HC Red No.10), 5-chloro-1,4-[di-(2,3-dihydroxypropyl)amino]-2-nitrobenzene (HC Red No. 11), 2-[(2-hydroxyethyl)amino]-4,6-dinitrophenol, 4-ethylamino-3-nitrobenzoic acid, 2-[(4-amino-2-nitrophenyl)-amino]benzoic acid, 2-chloro-6-ethylamino-4-nitrophenol, 2-amino-6-chloro-4-nitrophenol, 4-[(3-hydroxypropyl)amino]-3-nitrophenol, 2,5-diamino-6-nitropyridine, 1,2,3,4-tetrahydro-6-nitroquinoxaline, 7-amino-3,4-dihydro-6-nitro-2H-1,4-benzoxazine (HC Red No. 14), 1-amino-2-[(2-hydroxyethyl)amino]-5-nitrobenzene (HC Yellow No. 5), 1-(2-hydroxyethoxy)-2-[(2-hydroxyethyl)amino]-5-nitrobenzene (HC Yellow No. 4), 1-[(2-hydroxyethyl)amino]-2-nitrobenzene (HC Yellow No. 2), 2-[(2-hydroxyethyl)amino]-1-methoxy-5-nitrobenzene, 2-amino-3-nitrophenol, 1-(2-hydroxyeth-oxy)-3-methylamino-4-nitrobenzene, 2,3-(dihydroxypropoxy)-3-methylamino-4-nitrobenzene, 2-[(2-hydroxyethyl)amino]-5-nitrophenol (HC Yellow No. 11), 3-[(2-aminoethyl)amino]-1-methoxy-4-nitrobenzene hydrochloride (HC Yellow No. 9), 1-[(2-ureidoethyl)amino]4-nitrobenzene, 4-[(2,3-dihydroxypropyl)amino]-3-nitro-1-trifluoromethylbenzene (HC Yellow No. 6), 1-chloro-2,4-bis[(2-hydroxyethyl)amino]-5-nitrobenzene (HC Yellow No. 10), 4-[(2-hydroxyethyl)amino]-3-nitro-1-methylbenzene, 1-chloro-4-[(2-hydroxyethyl)amino]-3-nitrobenzene (HC Yellow No. 12), 4-[(2-hy-droxyethyl)amino]-3-nitro-1-trifluoromethylbenzene (HC Yellow No. 13), 4-[(2-hydroxyethyl)-amino]-3-nitrobenzonitrile (HC Yellow No. 14), 4-[(2-hydroxyethyl)amino]-3-nitrobenzamide (HC Yellow No. 15), 1,4-di[(2,3-dihydroxypropyl)amino]-9,10-anthraquinone, 1-[(2-hydroxyethyl)ami-no]-4-methylamino-9, 10-anthraquinone (C.I. 61505, Disperse Blue No. 3), 2-[(2-amino-ethyl)amino]-9,10-anthraquinone (HC Orange No. 5), 1 -hydroxy-4-[(4-methyl-2-sulfophenyl)-amino]-9,10-anthraquinone, 1 -[(3-aminopropyl)amino]-4-methylamino-9,10-anthraquinone (HC Blue No. 8), 1-[(3-aminopropyl)amino]-9,10-anthraquinone (HC Red No. 8), 1,4-diamino-2-methoxy-9,10-anthraquinone (C.I. 62015, Disperse Red No. 11, Solvent Violet No. 26), 1,4-dihydroxy-5,8-bis[(2-hydroxyethyl)amino]-9,10-anthraquinone (C.I. 62500, Disperse Blue No. 7, Solvent Blue No. 69), 9-(dimethylamino)benzo[a]-phenoxazin-7-ium chloride (C.I. 51175; Basic Blue No. 6), di[4-(diethylamino)phenyl][4-(ethylamino)naphthyl]carbenium chloride (C.I. 42595; Basic Blue No. 7), 3,7-di(dimethylamino)phenothiazin-5-ium chloride (C.I. 52015; Basic Blue No. 9), di[4-(dimethylamino)phenyl][4-(phenylamino)naphthyl]carbenium chloride (C.I. 44045; Basic Blue No. 26), 2-[(4-(ethyl-(2-hydroxyethyl)amino)phenyl)azo]-6-methoxy-3-methylbenzothiazolium methylsulfate (C.I. 11154; Basic Blue No. 41), 8-amino-2-bromo-5-hydroxy-4-imino-6-{[(3-trimethylammonio)phenyl]amino}-1(4H)-naphthalenone chloride (C.I. 56059; Basic Blue No. 99), bis[4-(dimethylamino)phenyl][4-(methylamino)phenyl]carbenium chloride (C.I. 42535; Basic Violet No. 1), tris[4-(dimethylamino)phenyl]carbenium chloride (C.I. 42555; Basic Violet No. 3), 2-[3,6-(diethylamino)dibenzopyranium-9-yl]benzoyl chloride (C.I. 45170; Basic Violet No. 10), di(4-ami-nophenyl)-(4-amino-3-methylphenyl)carbenium chloride (C.I. 42510; Basic Violet No. 14), 1,3-bis[(2,4-diamino-5-methylphenyl)azo]-3-methylbenzene (C.I. 21010; Basic Brown No. 4), 1-[(4-aminophenyl)azo]-7-(trimethylammonio)-2-naphthol chloride (C.I. 12250; Basic Brown No. 16), 1-[(4-amino-2-nitro-phenyl)azo]-7-(trimethylammonio)-2-naphthol chloride and 1-[(4-amino-3-nitrophenyl)azo]-7-(trimethylammonio)-2-naphthol chloride (1:1) (C.I. 12251; Basic Brown No. 17), 3,7-diamino-2,8-dimethyl-5-phenylphenazinium chloride (C.I. 50240; Basic Red. No. 2), 1,4-dimethyl-5-{[4-(dimethylamino)phenyl]azo}-1,2,4-triazolium chloride (C.I. 11055; Basic Red No. 22), 2-hydroxy-1-[(2-methoxyphenyl)azo]-7-(trimethylammonio)-naphthalene chloride (C.I. 12245; Basic Red No. 76), 2-{2-[(2,4-dimethoxyphenyl)amino]ethenyl}-1,3,3-trimethyl-3H-indol-1-ium chloride (C.I. 48055; Basic Yellow No. 11), 3-methyl-1-phenyl-4-{[3-(trimethylammonio)phenyl]azo}pyrazol-5-one chloride (C.I. 12719; Basic Yellow No. 57), bis[4-(diethylamino)phenyl]phenylcarbenium hydrogen sulfate (1:1) (C.I. 42040; Basic Green No. 1), 1-[di-(2-hydroxyethyl)amino]-3-methyl-4-[(4-nitrophenyl)azo]benzene (C.I. 11210, Disperse Red No. 17), 4-[(4-aminophenyl)azo]-1-[di-(2-hydroxyethyl)amino]-3-methylbenzene (HC Yellow No. 7), 2,6-diamino-3-[(pyridin-3-yl)-azo]pyridine, disodium 6-hydroxy-5-[(4-sulfophenyl)azo]-2-naphthalenedisulfonate (C.I. 15985; Food Yellow No. 3; FD&C Yellow No. 6), disodium 2,4-dinitro-1-naphthol-7-sulfonate (C.I. 10316; Acid Yellow No. 1; Food Yellow No. 1), 2-(indan-1,3-dion-2-yl)-quinolin-x,x-sulfonic acid (mixture of mono- and disulfonic acid) (C.I. 47005; D&C Yellow No. 10; Food Yellow No. 13; Acid Yellow No. 3), trisodium 5-hydroxy-1-(4-sulfophenyl)-4-[(4-sulfophenyl)azo]pyrazole-3-carboxylate (C.I. 19140; Food Yellow No. 4; Acid Yellow No. 23), 9-(2-carboxyphenyl)-6-hy-droxy-3H-xanthen-3-one (C.I. 45350; Acid Yellow No. 73; D&C Yellow No. 8), sodium 5-[(2,4-di-nitrophenyl)amino]-2-phenylaminobenzene-sulfonate (C.I. 10385; Acid Orange No. 3), mono-sodium 4-[(2,4-dihydroxyphenyl)-azo]benzenesulfonate (C.I. 14270; Acid Orange No. 6), sodium 4[(2-hydroxynaphth-1-yl)azo]benzenesulfonate (C.I. 15510; Acid Orange No. 7), sodium 4-[(2,4-dihydroxy-3-[(2,4-dimethylphenyl)azo]phenyl)azo]benzenesulfonate (C.I. 20170; Acid Orange No. 24), disodium 4-hydroxy-3-[(4-sulfonaphth-1-yl)azo]-1-naphthalenedisulfonate (C.I. 14720; Acid Red No. 14), trisodium 6-hydroxy-5-[(4-sulfonaphth-1-yl)azo]-2,4-naphthalenedisulfonate (C.I. 16255; Ponceau 4R; Acid Red No. 18), trisodium 3-hydroxy-4-[(4-sulfonaphth-1-yl)azo]-2,7-naphthalenedisulfonate (C.I.16185; Acid Red No. 27), disodium 8-amino-1-hydroxy-2-(phenylazo)-3,6-naphthalenedisulfonate (C.I. 17200; Acid Red No. 33), disodium 5-(acetylamino)4-hydroxy-3-[(2-methylphenyl)azo]-2,7-naphthalenedisulfonate (C.I. 18065; Acid Red No. 35), disodium 2-(3-hydroxy-2,4,5,7-tetraiododibenzopyran-6-on-9-yl)benzoate (C.I. 45430; Acid Red No. 51), N-[6-(diethylamino)-9-(2,4-disulfophenyl)-3H-xanthen-3-ylidene]-N-ethylethaneammonium hydroxide inner salt, sodium salt (C.I. 45100; Acid Red No. 52), disodium 8-{ [4-(phenylazo)phenyl]azo}-7-naphthol-1,3-disulfonate (C.I. 27290; Acid Red No. 73); 2′,4′,5′,7′-tetrabromo-3′,6′-dihydroxyspiro{isobenzofuran-1(3H),9′-[9H]xanthen}-3-one disodium salt (C.I. 45380; Acid Red No. 87), 2′,4′,5′,7′-tetrabromo4,5,6,7-tetrachloro-3′,6′-dihydroxyspiro-{isobenzofuran-1-3H),9′[9H]-xanthen}-3-one disodium salt (C.I. 45410; Acid Red No. 92), 3′,6′-dihydroxy-4′,5′-diiodo-spiro{isobenzofuran-1(3H),9(9H)-xanthen}-3-one disodium salt (C.I. 45425; Acid Red No. 95), (2-sulfophenyl)di[4-(ethyl((4-sulfophenyl)methyl)amino)phenyl]carbenium disodium salt, betaine (C.I. 42090; Acid Blue No. 9; FD&C Blue No. 1), 1,4-bis[(2-sulfo-4-methylphenyl)amino]-9,10-anthraquinone disodium salt (C.I. 61 570; Acid Green No. 25), bis[4-(dimethylamino)phenyl]-(3,7-disulfo-2-hydroxynaphth-1-yl)carbenium inner salt, monosodium salt (C.I. 44090; Food Green No. 4; Acid Green No. 50), bis[4-(diethyl-amino)phenyl](2,4-di-sulfophenyl)carbenium inner salt, sodium salt (2:1) (C.I. 42045; Food Blue No. 3; Acid Blue No. 1), bis[4-[diethylamino)phenyl](5-hydroxy-2,4-disulfophenyl)carbenium inner salt, calcium salt (2:1) (C.I. 42051; Acid Blue No. 3), sodium 1-amino-4-(cyclohexylamino)-9,10-anthraquinone-2-sulfonate (C.I. 62045; Acid Blue No. 62), disodium 2-(1,3-dihydro-3-keto-5-sulfo-2H-indol-2-ylidene)-2,3-dihydro-3-keto-1H-indol-5-sulfonate (C.I. 73015; Acid Blue No. 74), 9-(2-carboxy-phenyl)-3-[(2-methylphenyl)amino]-6-[(2-methyl-4-sulfophenyl)amino]xanthylium inner salt, mono-sodium salt (C.I. 45190; Acid Violet No. 9), 1-hydroxy-4-[(4-methyl-2-sulfophenyl)amino]-9,10-anthraquinone sodium salt (C.I. 60730; D&C Violet No. 2; Acid Violet No. 43), bis{3-nitro4-[(4-phenylamino)-3-sulfophenylamino]phenyl} sulfone (C.I. 10410; Acid Brown No. 13), disodium 5-amino-4-hydroxy-6-[(4-nitrophenyl)azo]-3-(phenylazo)-2,7-naphthalenedisulfonate (C.I. 20470; Acid Black No. 1), 3-hydroxy4-[(2-hydroxynaphth-1-yl)azo]-7-nitro-1-naphthalenesulfonic acid chromium complex (3:2) (C.I. 15711); Acid Black No. 52), disodium 3-[(2,4-dimethyl-5-sulfophenyl)azo]-4-hydroxy-1-naphthalenedisulfonate (C.I. 14700; Food Red No. 1; FD&C Red No. 4), tetrasodium 4-(acetylamino)-5-hydroxy-6-[(7-sulfo-4-[(4-sulfophenyl)azo]naphth-1-yl)azo]-1,7-naphthalenedisulfonate (C.I. 28440; Food Black No. 1) and sodium 3-hydroxy-4-(3-methyl-5-keto-1-phenyl-4,5-dihydro-1H-pyrazol-4-yl-azo)naphthalene-1-sulfonate, chromium complex (Acid Red No. 195), 3′,3″,4,5,5′,5″,6,7-octabromophenolsulfonphthalein (Tetrabromophenol Blue), 1-((4-amino-3,5-dimethylphenyl)-(2,6-dichlorophenyl)methylene)-3,5-dimethyl-4-imino-2,5-cyclohexadiene combined with phosphoric acid (1:1) (Basic Blue 77), 2′,4′,5′,7′-tetrabromo-4,5,6,7-tetrachloro-3′,640 -dihydroxyspiro{isobenzofuran-1-(3H),9′[9H]xanthen}-3-one disodium salt (Acid Red No. 92), N,N-di(2-hydroxyethyl)-3-methyl-4-[(4-nitrophenyl)azo]aniline (Disperse Red 17), disodium 2,4-dinitro-1-naphthol-7-sulfonate (Acid Yellow 1), sodium 4-[(2-hydroxynaphthalen-1-yl)azo]-benzenesulfonate (Acid Orange 7), 2-((4-(ethyl-(2-hydroxyethyl)amino)-2-methylphenyl)azo)-5-nitro-1,3-thiazole (Disperse Blue 106), 2,4-dinitro-1-naphthol, 2-[(4-aminophenyl)azo]-1,3-dimeth-yl-1H-imidazol-3-ium chloride, 1-methyl-4-[(methylphenylhydrazono)methyl]pyridinium methosulfate, 2-{[4-(dimethylamino)phenyl]azo}-1,3-dimethylimidazolium chloride, 2-((4-((4-methoxyphenyl)amino)phenyl)azo)-1,3-dimethyl-1H-imidazol-3-ium chloride and 1,3-dimethyl-2-((4-((phenylmethyl)-amino)phenyl)azo)-1H-imidazol-3-ium chloride, or in mixtures thereof.

Particularly preferred among the aforesaid direct dyes are the following compounds, alone or in combination with one another: hydroxyethyl-2-nitro-p-toluidine, 2-hydroxyethylpicramic acid, 4-nitrophenylaminourea, tri(4-amino-3-methylphenyl)carbenium chloride (Basic Violet 2), 1,4-diamino-9,10-anthracenedione (Disperse Violet 1), 1-(2-hydroxy-ethyl)amino-2-nitro4-[di(2-hydroxyethyl)amino]benzene (HC Blue No. 2), 4-[ethyl-(2-hydroxyethyl)amino]-1-[(2-hydroxyethyl)amino]-2-nitrobenzene hydrochloride (HC Blue No. 12), 1-amino-4-[di(2-hydroxyethyl)amino]-2-nitrobenzene hydrochloride (HC Red No. 13), 4-amino-1-[(2-hydroxyethyl)amino]-2-nitrobenzene (HC Red No. 3), 4-amino-3-nitrophenol, 4-[(2-hydroxyethyl)amino]-3-nitrophenol, 1-amino-5-chloro-4-[(2,3-dihydroxypropyl)amino]-2-nitrobenzene (HC Red No. 10), 5-chloro-1,4-[di(2,3-dihydroxypropyl)amino]-2-nitrobenzene (HC Red No. 11), 2-chloro-6-ethylamino-4-nitrophenol, 2-amino-6-chloro-4-nitrophenol, 4-[(2-hydroxyethyl)amino]-3-nitro-1-trifluoromethylbenzene (HC Yellow No. 13), 8-amino-2-bromo-5-hydroxy-4-imino-6-{[3-(trimethylammonio)-phenyl]amino}-1(4H)-naphthalenone chloride (C.I. 56059; Basic Blue No. 99), 1-[(4-aminophenyl)azo]-7-(trimethylammonio)-2-naphthol chloride (C.I. 12250; Basic Brown No. 16), 1-[(4-amino-2-nitrophenyl)azo]-7-(trimethylammonio)-2-naphthol chloride (Basic Brown No. 17), 2-hydroxy-1-[(2-methoxyphenyl)azo]-7-(trimethylammonio)naphthalene chloride (C.I. 12245; Basic Red No. 76), 3-methyl-1-phenyl-4-{[3-(trimethylammonio)phenyl]azo}pyrazol-5-one chloride (C.I. 12719; Basic Yellow No. 57) and 2,6-diamino-3-[(pyridin-3-yl)azo]pyridine as well as the salts thereof.

The total quantity of direct dyes in the pellets of the invention amounts to 0.1 wt. % to 90 wt. % and preferably 1 wt. % to 70 wt. %.

Other known and common dyes used in hair colorants are described in, among other publications, E. Sagarin “Cosmetics, Science and Technology,” Interscience Publishers Inc., New York (1957), pages 503 ff, in H. Janistyn, “Handbuch der Kosmetika und Riechstoffe” [Manual of Cosmetics and Fragrances], vol. 3 (1973), pages 388 ff, and in K. Schrader “Grundlagen und Rezepturen der Kosmetika” [Fundamentals and Formulations of Cosmetics], 2nd edition (1989), pages 782-815, the disclosures.

The dye-containing pellets of the invention have many advantages. They are absolutely dust-free and free-flowing, but do not have the drawbacks presented by the common dust-free, oil-treated powders and, in particular, do not exert an adverse effect on product performance. Depending on the kind and layer thickness of the selected coating material, the release of the dyes is possible at any desired point in time (delayed release). Also possible is the use of reactive dyes together with reactive raw materials (for example, oxidants such as the persulfates and hydrogen peroxide salts or hydrogen peroxide addition products) as well as a clearly improved storage stability compared to powders and aqueous/alcoholic systems. Of particular interest is the possibility of uncomplicated achievement of multicolor effects through the kind and layer thickness of the selected coating material, whereas according to the current state of the art such multicolor effects can be attained only by extremely time-consuming and complicated techniques, for example, by sheet or strand techniques. In addition, the pellets of the invention have clearly better dosability than do prior-art colorants (powders, aqueous/alcoholic systems).

Other objects of the present invention, therefore, are the use of the aforesaid pellets for producing colorants for keratin fibers and an agent for coloring keratin fibers, for example hair and particularly human hair, which is prepared by mixing the aforesaid pellets with an aqueous or aqueous-alcoholic preparation.

The aqueous or aqueous-alcoholic preparation used can be either water or a mixture of water and a C₁-C₆ alcohol (for example, ethanol or isopropanol) or a common hydrogen peroxide solution or hydrogen peroxide emulsion or else a common hair cleaning agent, hair conditioner or hair-firming agent.

The composition of such preparations is known and can be found in pertinent text books on cosmetics, for example, in Karlheinz Schrader “Grundlagen und Rezepturen der Kosmetika” [Fundamentals and Formulations of Cosmetics], 2nd edition (1989).

For example, the colorant of the invention can contain antioxidants, for example, ascorbic acid, thioglycolic acid or sodium sulfite, as well as complexing agents for heavy metals, for example, an ethylenediaminetetraacetate or nitriloacetic acid, in an amount of up to 0.5 wt. %. Perfume oils can be contained in the dye carrier composition of the invention in an amount of up to 1 wt. %. Moreover, the aforedescribed hair colorant can optionally contain other auxiliary agents and additives commonly used in such colorants, for example, thickeners, for example, homopolymers of acrylic acid, vegetable gums, algal polysaccharides, amphiphilic associative thickeners; furthermore preservatives; complexing agents; wetting agents or emulsifiers from the classes of anionic, cationic, amphoteric or nonionic surface-active sub-stances; alkalizing agents (for example, ammonium salts, or amino acids such as glycine and alanine) furthermore hair-care agents, such as cationic polymers or resins, lanolin derivatives, cholesterol, vitamins, pantothenic acid and betaine. The said constituents are employed in amounts normally used for such purposes, for example, the wetting agents and emulsifiers are present at a concentration from 0.1 wt. % to 30 wt. % and the hair-care agents at a concentration from 0.1 wt. % to 5.0 wt. %.

The colorants containing oxidation dye precursors are used in combination with one or more known chemical oxidants, for example, hydrogen peroxide or a salt or adduct thereof, as well as persulfates such as sodium persulfate, potassium persulfate or ammonium persulfate, or they are activated by air oxidation (optionally in the presence of appropriate enzymes or catalysts). If simultaneous brightening and coloring of the fibers is desired, it is also possible to use colorants based on direct dyes—provided said dyes are sufficiently oxidation-resistant—in combination with one or more known oxidants, for example, hydrogen peroxide or a salt or adduct thereof as well as persulfates such as sodium persulfate, potassium persulfate, or ammonium persulfate.

To attain simultaneous brightening of the fibers, the dye-containing pellets of the invention can also contain an ammonium carbonate, for example, ammonium hydrogen carbonate, or an amino acid or a salt thereof, for example, sodium glycinate.

The pellets according to the invention offer an increased shine of the keratin fibers (up to 66%), a reduction of the scalp coloring of hair dyes, an improvement of the protection for the cortex, an enhancement of the colorant efficiency and a reduction of the load of colorants (up to 50%), and an improvement of the wash resistance.

The following examples will explain the subject matter of the invention in greater detail without limiting its scope to these examples.

### EXAMPLES

### Example 1

### Preparation of Dye Pellets by the Top Spray Method

In a Glatt fluidized bed granulator and coater, the following mixture A is heated to a product temperature of 34° C. with air at an incoming air temperature of 90° C. and an air flow rate of 18 m³/h.

Mixture A

| | |
|---|---|
| 381.2 g | of 4-(2-hydroxyethylamino)-3-nitrophenol |
| 101.0 g | of 2-[(2-hydroxyethyl)amino]-4,6-dinitrophenol |
| 100.0 g | of corn starch |

A 20% aqueous polyvinylpyrrolidone solution (“spraying solution”) is then sprayed onto this mixture at an initial spraying rate of 8 g/min. and at a spraying air pressure of 0.5 bar. In the course of the granulation process, the spraying rate is increased to 12 g/min. and the incoming air temperature is increased to 100° C., while the air flow rate is raised to a maximum of 30 m³/h. The product temperature is kept at 30° C. to 31° C. throughout the entire procedure. After 310 g has been sprayed on, the pellets are dried at a maximum product temperature of 57° C. and then cooled to 30° C. and screened.

### Example 2

### Preparation of Dye Pellets by Extruder Technology

Mixture A

| | |
|---|---|
| 1896 g | of 4-(2-hydroxyethylamino)-3-nitrophenol |
| 504 g | of 2-[(2-hydroxyethyl)amino]-4,6-dinitrophenol |
| 800 g | of microcrystalline cellulose |
| 800 g | of corn starch |

Mixture A is mixed in a vertical granulator (rotor rotational speed=150 rpm; chopper rotational speed=1000 rpm) for 1 minute and then, by means of a dual nozzle and with continuing mixing, is sprayed with 2091 g of a 6% aqueous hydroxypropylmethylcellulose solution. The composition thus obtained is extruded from a BR 200-type extruder (rotational speed=27 rpm; screen mesh diameter: 1.0 mm) at a product temperature of 30° C. The material thus obtained is then rounded in a P 50-type pelletizer for 1 minute at 550 rpm and then is dried in a Glatt vertical granulator at an incoming air temperature of 70° C., an air flow rate of 60 m³/h to 90 m³/h and a maximum product temperature of 51° C.

In a Glatt fluidized bed granulator and coater, 1500 g of the dried dye pellets is heated at an incoming air temperature of 50° C. and an air flow rate of 75 m³/h to a product temperature of 39° C. to 40° C. The pellets are then sprayed with a 10% aqueous solution of hydroxy-propylmethylcellulose at a spraying rate of 5 g/min and a spraying air pressure of 2.5 bar. In the course of the procedure, the spraying rate is increased to 8.5 g/min. After 2215 g of the spraying solution has been applied, corresponding to an addition of 14% of solids, the material is again dried at a maximum product temperature of 51° C. (incoming air temperature=70° C.), and then cooled to 27° C. and screened.

(Alternatively the drying and coating or the granulation, drying and coating can be carried out in the same processing step).

### Example 3

### Preparation of Dye Pellets by Extruder Technology

### Gold Color Shade

Mixture A

| | |
|---|---|
| 2400 g | of 2-amino-6-chloro-4-nitrophenol |
| 800 g | of microcrystalline cellulose |
| 800 g | of corn starch |

Mixture A is pelletized as in Example 2, but by using a 5.6% aqueous hydroxypropyl-methylcellulose solution as the coating agent.

### Example 4

### Preparation of Dye Pellets by Extruder Technology

### Red/Mahogany Color Shade

Mixture A

| | |
|---|---|
| 1896 g | of 3-nitro-p-hydroxyethylaminophenol |
| 504 g | of 2-hydroxyethylpicramic acid |
| 800 g | of microcrystalline cellulose |
| 800 g | of potato starch |

The preparation is carried out as described in Example 2, but by using a 6.25% aqueous hydroxymethylcellulose solution as the coating agent.

### Example 5

### Preparation of Dye Pellets by Extruder Technology

Mixture A

| | |
|---|---|
| 1411 g | of 2,5-diaminotoluene sulfate |
| 636 g | of 4-amino-2-hydroxytoluene |
| 353 g | of 2-amino-4-(β-hydroxyethylamino)anisole sulfate |
| 794 g | of ascorbic acid |
| 1058 g | of sodium sulfite |
| 800 g | of hydroxypropylcellulose |
| 1300 g | of corn starch |

The preparation is carried out as described in Example 2, but by using a 5.625% aqueous hydroxypropylmethylcellulose solution as the coating agent.

### Example 6

### Preparation of Dye Pellets by the Top Spray Method

Mixture A

| | |
|---|---|
| 7.2 g | of 5-amino-2-methylphenol |
| 16.0 g | of 2,5-diaminotoluene sulfate |
| 4.0 g | of 2-amino-4-(β-hydroxyethylamino)anisole sulfate |
| 3.0 g | of ascorbic acid |
| 4.0 g | of sodium sulfite |
| 965.8 g | of hydrolyzed corn starch (oligosaccharide) |

The mixture is pelletized as described in Example I with 563g of a 20% aqueous polyvinylpyrrolidone solution.

### Example 7

### Preparation of Dye Pellets by the Top Spray Method

Mixture A

| | |
|---|---|
| 879 g | of dextrose |

Mixture B: (dispersion)

| | |
|---|---|
| 30 g | of 2-amino-6-chloro-4-nitrophenol |
| 500 g | of a 20% aqueous polyvinylpyrrolidone solution |
| | solution (mol. wt. = 30,000 g/mol) |

The mixture is pelletized as described in Example 1, but by using the aforedescribed mixture (B) as spraying solution.

### Example 8

### Preparation of Dye Pellets by the Top Spray Method

Mixture A:

| | |
|---|---|
| 693.7 g | of dextrose |

Mixture B: (dispersion)

| | |
|---|---|
| 20.8 g | of 4-(β-hydroxyethylamino)-3-nitrophenol |
| 5.5 g | of 2-hydroxyethylpicramic acid |
| 400.0 g | of 20% aqueous polyvinylpyrrolidone solution |
| | (mol. wt. = 30,000 g/mol) |

The mixture is pelletized as described in Example 1, but by using the aforedescribed mixture (B) as spraying solution.

### Example 9

### Preparation of Dye Pellets by the Top Spray Method

Mixture A:

| | |
|---|---|
| 721.6 g | of dextrose |

Mixture B: (dispersion)

| | |
|---|---|
| 17.0 g | of 2,5-diaminotoluene sulfate |
| 2.0 g | of resorcinol |
| 7.6 g | of 2-methylresorcinol |
| 2.2 g | of 2-amino-6-chloro-4-nitrophenol |
| 2.4 g | of 6-amino-m-cresol |
| 0.2 g | of 4-amino-2-hydroxytoluene |
| 3.0 g | of ascorbic acid |
| 4.0 g | of sodium sulfite |
| 80.0 g | of alanine |
| 60.0 g | of glycine |
| 500.0 g | of 20% aqueous polyvinylpyrrolidone solution |
| | (mol. wt. = 30,000 g/mol) |

Mixture (A) is pelletized as described in Example 1, but by using the aforedescribed Mixture (B) as spraying solution.

### Example 10

### Creamy Hair Colorant

Cream base

| | | |
|---|---|---|
| 8.70 | g | of cetylstearyl alcohol |
| 2.30 | g | of glyceryl stearate (self-emulsifying) |
| 0.80 | g | of lanolin |
| 3.80 | g | of lanolin alcohol |
| 1.42 | g | of sodium cetylstearylsulfate |
| 0.07 | g | of formaldehyde |
| 0.01 | g | of tocopherol |
| 0.20 | g | of perfume |
| 10.00 | g | of ammonia |
| to 100.00 | g | water |

The aforedescribed cream base is prepared by the conventional hot-emulsification method. Before use, it is mixed with the dye pellets in an appropriate ratio as in one of Examples 1, 2, 3, 7, or 8 in a dye cup or shaking bottle.

### Example 11

### Oxidation Hair Colorant

Hydrogen Peroxide Emulsion

| | | |
|---|---|---|
| 9.00 | g | of hydrogen peroxide |
| 1.80 | g | of cetylstearyl alcohol |
| 3.30 | g | of polyvinylpyrrolidone-styrene copolymer |
| 0.20 | g | of disodium phosphate |
| 0.20 | g | of sodium laurylsulfate |
| 0.10 | g | of salicylic acid |
| 0.08 | g | of phosphoric acid |
| to 100.00 | g | water |

The above-described hydrogen peroxide emulsion is prepared by the conventional hot-emulsification method. Just before use, this hydrogen peroxide emulsion is mixed with dye pellets as in Example 5, 6, or 9 in a dye cup or a shaking flask.

Unless otherwise indicated, all percentages are by weight.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A dye-containing pellet, which is obtained by homogeneous mixing of at least one natural or synthetic dye-containing starting material with a suitable carrier material, followed by coating with a suitable encapsulation material.

2. A dye-containing pellet, which is obtained by coating a suitable carrier material with a mixture of at least one natural or synthetic dye and at least one suitable encapsulation material.

3. A pellet as defined in claim 1, wherein the carrier material is selected from the group consisting of polyvinylpyrrolidones, dextrose, oligosaccharides, microcrystalline cellulose derivatives, physically or chemically modified starches or starch derivatives, synthetic calcium silicate, diatomite, silicon dioxide, and other free-flowing, non-baking powders.

4. A pellet as defined in claim 3, wherein the carrier material is selected from the group consisting of polyvinylpyrrolidone, dextrose, oligosaccharides, hydroxypropylmethylcellulose, methylcellulose, hydroxyethylcellulose, hydroxypropylcellulose, nonoxynol-hydroxyethylcellulose, cetylhydroxyethylcellulose and nonionic starch derivatives modified with propylene oxide.

5. A pellet as defined in claim 1, wherein the encapsulating material is selected from the group consisting of cellulose derivatives, polyethylene dispersions, polyacrylic acids, polyvinyl alcohols, polyvinylpyrrolidones, polycarbonates, polyesters, polyamides or natural film-formers.

6. A pellet as defined in claim 5, wherein the natural film-former is selected from among chitosan, shellac, oligosaccharides, or colophony.

7. A pellet as defined in claim 1, wherein the dye is selected from the group consisting of oxidation dye precursors and direct dyes.

8. A pellet as defined in claim 1, wherein the dye is selected from the group consisting of 2,5-diaminotoluene, 2,4-diaminophenoxyethanol, resorcinol, 2-methyl-resorcinol, m-aminophenol, 4-amino-m-cresol, 4-amino-2-hydroxytoluene, 6-amino-m-cresol, 2-amino-4-hydroxyethylaminoanisol, 1-naphthol, hydroxyethyl-3,4-methylenedioxyaniline, 2,5-diaminophenylethanol, N,N-bis(2-hydroxyethyl)-p-phenylenediamine, phenylmethylpyrazolone, 1-hydroxyethyl-4,5-diaminopyrazole and 2-amino-6-chloro-4-nitro-phenol, or the salts thereof.

9. A pellet as defined in claim 1, wherein the dye is selected from the group consisting of hydroxyethyl-2-nitro-p-toluidine, 2-hydroxyethylpicramic acid, 4-nitrophenylaminourea, Basic Violet 2, Disperse Violet 1, HC Blue No. 2, HC Blue No. 12, HC Red No. 13, HC Red No. 3,4-amino-3-nitrophenol, 4-[(2-hydroxyethyl)amino]-3-nitrophenol, HC Red No. 10, HC Red No. 11, 2-chloro-6-ethylamino-4-nitrophenol, 2-amino-6-chloro-4-nitrophenol, HC Yellow No. 13, Basic Blue No. 99, Basic Brown No. 16, Basic Brown No. 17, Basic Red No. 76, Basic Yellow No. 57 and 2,6-diamino-3-[(pyridin-3-yl)azo]pyridine, and the salts thereof.

10. A pellet as defined in claim 2, wherein the carrier material is selected from the group consisting of polyvinylpyrrolidones, dextrose, oligosaccharides, microcrystalline cellulose derivatives, physically or chemically modified starches or starch derivatives, synthetic calcium silicate, diatomite, silicon dioxide, and other free-flowing, non-baking powders.

11. A pellet as defined in claim 10, wherein the carrier material is selected from the group consisting of polyvinylpyrrolidone, dextrose, oligosaccharides, hydroxypropylmethylcellulose, methylcellulose, hydroxyethylcellulose, hydroxypropylcellulose, nonoxynol-hydroxyethylcellulose, cetylhydroxyethylcellulose, and nonionic starch derivatives modified with propylene oxide.

12. A pellet as defined in claim 2, wherein the encapsulating material is selected from the group consisting of cellulose derivatives, polyethylene dispersions, polyacrylic acids, polyvinyl alcohols, polyvinylpyrrolidones, polycarbonates, polyesters, polyamides, or natural film-formers.

13. A pellet as defined in claim 12, wherein the natural film-former is selected from among chitosan, shellac, oligosaccharides, or colophony.

14. An agent for coloring keratin fibers obtained by mixing at least one dye-containing pellet as defined in claim 1 with an aqueous or aqueous-alcoholic preparation.

15. An agent as defined in claim 14, wherein the aqueous or aqueous-alcoholic preparation is selected from the group consisting of water, mixtures of water and C₁-C₆ alcohols, hydrogen peroxide solutions or hydrogen peroxide emulsions, hair cleaning agents, hair conditioners and hair-firming agents.

16. An agent for coloring keratin fibers obtained by mixing at least one dye-containing pellet as defined in claim 2 with an aqueous or aqueous-alcoholic preparation.

17. An agent as defined in claim 16, wherein the aqueous or aqueous-alcoholic preparation is selected from the group consisting of water, mixtures of water and C₁-C₆ alcohols, hydrogen peroxide solutions or hydrogen peroxide emulsions, hair cleaning agents, hair conditioners and hair-firming agents.

18. A method for increasing the shine of the keratin fibers wherein an agent for coloring keratin fibers obtained by mixing at least one dye-containing pellet as defined in claim 1 with an aqueous or aqueous-alcoholic preparation is applied to the keratin fiber in an amount adequate for the coloring of the keratin fibers, the coloring agent is left to act at 15° C. to 45° C. for 1 minute to 60 minutes, and the keratin fiber is then thoroughly rinsed with water, and finally dried.

19. A method for reducing the scalp coloring of hair dyes wherein an agent for coloring hair obtained by mixing at least one dye-containing pellet as defined in claim 1 with an aqueous or aqueous-alcoholic preparation is applied to the hair in an amount adequate for the coloring of the hair, the hair dye is left to act at 15° C. to 45° C. for 1 minute to 60 minutes, and the hair is then thoroughly rinsed with water, and finally dried.

20. A method for improving the protection for the cortex wherein an agent for coloring hair obtained by mixing at least one dye-containing pellet as defined in claim 1 with an aqueous or aqueous-alcoholic preparation is applied to the hair in an amount adequate for the coloring of the hair, the hair dye is left to act at 15° C. to 45° C. for 1 minute to 60 minutes, and the hair is then thoroughly rinsed with water, and finally dried.

21. A method for increasing the colorant efficiency and reducing the load of colorants of hair dyes wherein an agent for coloring hair obtained by mixing at least one dye-containing pellet as defined in claim 1 with an aqueous or aqueous-alcoholic preparation is applied to the hair in an amount adequate for the hair coloring, the coloring agent is left to act at 15° C. to 45° C. for 1 minute to 60 minutes, and the hair is then thoroughly rinsed with water, and finally dried.

22. A method for improving the wash resistance of dyed hair wherein an agent for coloring hair obtained by mixing at least one dye-containing pellet as defined in claim 1 with an aqueous or aqueous-alcoholic preparation is applied to the hair in an amount adequate for the hair coloring, the coloring agent is left to act at 15° C. to 45° C. for 1 minute to 60 minutes, and the hair is then thoroughly rinsed with water, and finally dried.

23. A method for increasing the shine of the keratin fibers wherein an agent for coloring keratin fibers obtained by mixing at least one dye-containing pellet as defined in claim 2 with an aqueous or aqueous-alcoholic preparation is applied to the keratin fiber in an amount adequate for the coloring of the keratin fibers, the coloring agent is left to act at 15° C. to 45° C. for 1 minute to 60 minutes, and the keratin fiber is then thoroughly rinsed with water, and finally dried.

24. A method for reducing the scalp coloring of hair dyes wherein an agent for coloring hair obtained by mixing at least one dye-containing pellet as defined in claim 2 with an aqueous or aqueous-alcoholic preparation is applied to the hair in an amount adequate for the coloring of the hair, the hair dye is left to act at 15° C. to 45° C. for 1 minute to 60 minutes, and the hair is then thoroughly rinsed with water, and finally dried.

25. A method for improving the protection for the cortex wherein an agent for coloring hair obtained by mixing at least one dye-containing pellet as defined in claim 2 with an aqueous or aqueous-alcoholic preparation is applied to the hair in an amount adequate for the coloring of the hair, the hair dye is left to act at 15° C. to 45° C. for 1 minute to 60 minutes, and the hair is then thoroughly rinsed with water, and finally dried.

26. A method for increasing the colorant efficiency and reducing the load of colorants of hair dyes wherein an agent for coloring hair obtained by mixing at least one dye-containing pellet as defined in claim 2 with an aqueous or aqueous-alcoholic preparation is applied to the hair in an amount adequate for the hair coloring, the coloring agent is left to act at 15° C. to 45° C. for 1 minute to 60 minutes, and the hair is then thoroughly rinsed with water, and finally dried.

27. A method for improving the wash resistance of dyed hair wherein an agent for coloring hair obtained by mixing at least one dye-containing pellet as defined in claim 2 with an aqueous or aqueous-alcoholic preparation is applied to the hair in an amount adequate for the hair coloring, the coloring agent is left to act at 15° C. to 45° C. for 1 minute to 60 minutes, and the hair is then thoroughly rinsed with water, and finally dried.
